(19) United States
(12) Patent Application Publication
(10) Pub. No.: US 2021/0161725 A1
(43) Pub. Date: Jun. 3, 2021

(54) WOUND DRESSING SYSTEM FOR MANAGEMENT OF FLUIDS IN A WOUND AND METHODS FOR MANUFACTURING SAME

(71) Applicant: KCI LICENSING, INC., San Antonio, TX (US)

(72) Inventors: Thomas EDWARDS, Wimborne (GB); Justin Alexander LONG, Lago Vista, TX (US); Christopher Brian LOCKE, Bournemouth (GB)

(21) Appl. No.: 17/265,887

(22) PCT Filed: Aug. 6, 2019
(86) PCT No.: PCT/US2019/045216

§ 371 Date: Feb. 4, 2021

Related U.S. Application Data
(60) Provisional application No. 62/717,125, filed on Aug. 10, 2018.

(51) Int. Cl. A61F 13/02; A61F 13/00; A61M 1/00

(52) U.S. Cl. CPC A61F 13/0216; A61M 1/0088; A61F 13/00068

(57) ABSTRACT

A fluid management bridge for providing a fluid communication between a wound interface dressing and a negative pressure source is disclosed. The fluid management bridge comprises a first end having a first fluid port configured to be coupled to a port of the wound interface dressing and a second end having a second fluid port configured to be coupled to the negative pressure device. The first end of the fluid management bridge is configured to provide for central alignment of the first end with the port of the wound interface dressing.

### RELATED APPLICATIONS

The present application claims priority to U.S. Patent Provisional Application No. 62/717,125, filed Aug. 10, 2018, which is herein incorporated by reference in its entirety.

### FIELD

The present disclosure generally relates to a wound therapy system and, more particularly to an improved wound therapy system and method in which alignment features are used to facilitate establishing a connection among various portions of the wound therapy system.

### BACKGROUND

Chronic, acute, or complex wounds, such as venous leg ulcers (VLU), can result in various negative effects on the health and well-being of patients. Patients suffering from such wounds can often experience physical symptoms such as pain, immobility, and lack of energy. Combination therapy methods, such as negative pressure wound therapy (NPWT) and compression garments (e.g., compression bandages, garments, and stockings) are often used to promote healing of such wounds.

Negative pressure wound therapy (also known as “reduced pressure therapy,” or “vacuum therapy”) commonly involves application of reduced pressure in proximity to a tissue site augments to accelerate growth of new tissue. This treatment provides a number of benefits, including faster healing and increased formulation of granulation tissue. Reduced pressure is typically applied to the tissue through a wound insert (e.g., a porous pad or other manifold device). The wound insert typically contains cells or pores that are capable of distributing reduced pressure to the tissue and channeling fluids that are drawn from the tissue. The wound insert can be incorporated into a wound dressing. The wound dressing can, in turn, include other components that facilitate treatment (e.g., a drape, such as an adhesive surgical drape). Further, fluid irrigation can be used in conjunction with negative pressure wound therapy to promote healing.

Although NPWT has been highly successful in the promotion of wound closure, NPWT wound dressings and their connection to an NPWT device can be difficult and time-consuming to use (e.g., due at least in part to the number of connections which must be made between wound dressings, connection pads, and conduits to the NPWT device).

### SUMMARY

In one aspect, a wound dressing system is described. The wound dressing system comprises a base wound interface, configured to be disposed on a portion of a patient's skin, and a fluid management bridge. The fluid management bridge includes a first end configured to be coupled to the base wound interface and a second end configured to connect to a negative pressure source, and the first end of the fluid management bridge includes one or more alignment features configured to be aligned to corresponding alignment features on the base wound interface.

In another aspect, a fluid management bridge for providing a fluid communication between a wound interface dressing and a negative pressure source is disclosed. The fluid management bridge comprises a first end having a first fluid port configured to be coupled to a port of the wound interface dressing and a second end having a second fluid port configured to be coupled to the negative pressure device. The first end of the fluid management bridge is configured to provide for central alignment of the first end with the port of the wound interface dressing.

In yet another aspect, a method for extracting fluid from a base wound interface is disclosed. The disclosed method comprises aligning one or more alignment features surrounding a first port disposed on a first end of a fluid management bridge with corresponding alignment features surrounding a fluid port of the base wound interface, establishing a connection between the first port and the fluid port, and extracting the fluid from the base wound interface, through the fluid port of the base wound interface and the first port of the fluid management bridge.

In another aspect, a method for manufacturing a fluid management bridge configured to provide a fluid communication between a wound interface dressing and a negative pressure source is disclosed. The claimed method comprises disposing one or more wicking layers configured to transport the fluid through the fluid management bridge on a layer of absorbent material, forming an outer layer of polyurethane film on the one or more wicking layers, forming a first fluid port on a first side of the one or more wicking layers, the first port being configured for connecting to a port of the wound interface dressing, forming a second fluid port on a second side of the one or more wicking layers, the second port being configured for connecting to the negative pressure device, and forming one or more alignment features on the first side of the one or more wicking layers. The alignment features are at least one of: disposed adjacent to the first fluid port or dimensionally linked to the first fluid port, and the alignment features are configured for alignment with corresponding alignment features disposed adjacent to or dimensionally linked to the port of the wound interface dressing.

In other examples, any of the aspects above, or any system, method, apparatus described herein can include one or more of the following features.

The fluid management bridge can be configured to provide a fluid communication between the base wound interface and the negative pressure device. Further, the first end of the fluid management bridge can include a first port configured to be coupled to a fluid port on the base wound interface and the second end of the fluid management bridge can include a second port configured to be coupled to the negative pressure device. Furthermore, the one or more alignment features on the fluid management bridge are disposed adjacent to the first port. Additionally or alternatively, the one or more alignment features on the fluid management bridge are dimensionally linked to the first port. In some embodiments, the one or more alignment features on the fluid management bridge comprise one or more lines, wherein each line can be perpendicular to a central axis line of the first port.

In some embodiments, the corresponding alignment features on the base wound interface can be disposed adjacent to the fluid port of the base wound interface. Additionally or alternatively, the corresponding alignment features on the base wound interface can be dimensionally linked to the fluid port. The corresponding alignment features on the base wound interface can comprise one or more lines, arranged such that each line can be an extension of a central axis line of the fluid port. Further, at least one of the one or more alignment features of the fluid management bridge or the corresponding alignment features of the base wound interface can comprise one or more geometrical shapes including at least one of: one or more circular shapes, one or more polygonal shapes, or a combination thereof.

The base wound interface can comprise a fluid port configured to connect the base wound interface to the negative pressure source. Further, the base wound interface can comprises a fluid permeable base layer configured to be disposed on the portion of the patient's skin. Additionally or alternatively, the base wound interface can comprises two or more wicking layers that configured to transport fluid through the base wound interface. Further, the base wound interface can comprise an adhesive layer configured to affix the base wound interface to the patient's skin. In some embodiments, the corresponding alignment features can be included on the adhesive layer of the base wound interface. Additionally or alternatively, the corresponding alignment features can be included on the adhesive layer by at least one of: applying a biocompatible ink, printing onto the adhesive layer of the base wound interface, embossing onto the base wound interface, or laminating on the base wound interface.

The fluid management bridge can comprise an outer layer, a middle layer, and an inner layer. The one or more alignment features can be included on the outer layer of the fluid management bridge. Additionally or alternatively, the one or more alignment features can be included on the first end, over the outer layer of the fluid management bridge by at least one of: applying a biocompatible ink, printing onto the outer layer, embossing onto the outer layer, or laminating on the outer layer.

The outer layer of the fluid management bridge can comprise a polyurethane film. Further, the middle layer can comprise one or more wicking layers configured to transport fluid through the fluid management bridge. Furthermore, the inner layer can comprise an absorbent material. In some embodiments, the absorbent material can be configured to store fluid within the fluid management bridge. The one or more alignment features can be included on at least one of the middle layer or the inner layer of the fluid management bridge.

In some embodiments, the first end of the fluid management bridge can be geometrically shaped to provide for the central alignment of the first end with the port of the wound interface dressing. For example, the first end of the fluid management bridge can comprise a semi-circular geometrical structure configured to centrally align with one or more alignment features on the base wound interface.

A second port disposed on a second end of the fluid management bridge can be connected to a negative pressure device. Any fluid extracted from the base wound interface can be transferred to the negative pressure device through the fluid management bridge. In some embodiments, fluid extracted from the base wound interface can be stored in the fluid management bridge.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention. The dimensions of the various features are arbitrarily expanded or reduced for clarity.

FIG. 1 illustrates a high-level view of a therapy system according to some embodiments disclosed herein.

FIG. 2 is an illustrative example of a bridge assembly suitable for use with the therapy system and dressing shown in FIG. 1.

FIG. 3A illustrates a base wound dressing according to some embodiments disclosed herein.

FIG. 3B illustrates a fluid management bridge according to some embodiments disclosed herein.

FIG. 4A illustrates a base wound dressing according to some embodiments disclosed herein.

FIG. 4B illustrates a fluid management bridge according to some embodiments disclosed herein.

FIG. 5A illustrates a base wound dressing according to some embodiments disclosed herein.

FIG. 5B illustrates a fluid management bridge according to some embodiments disclosed herein.

FIG. 5C illustrates a base wound dressing according to some embodiments disclosed herein.

FIG. 6A graphically depicts an example of various layers that can be incorporated in a base wound dressing according to some embodiments disclosed herein.

FIG. 6B graphically depicts an example of various layers that can be incorporated in a fluid management bridge according to some embodiments disclosed herein.

FIG. 7A illustrates a base wound dressing according to some embodiments disclosed herein.

FIG. 7B illustrates a fluid management bridge according to some embodiments disclosed herein.

FIG. 7C graphically depicts a fluid management bridge as connected to a base wound dressing.

FIG. 7D graphically depicts another fluid management bridge as connected to a base wound dressing.

FIG. 8 graphically illustrates a wound therapy system according to some embodiments disclosed herein.

### DETAILED DESCRIPTION

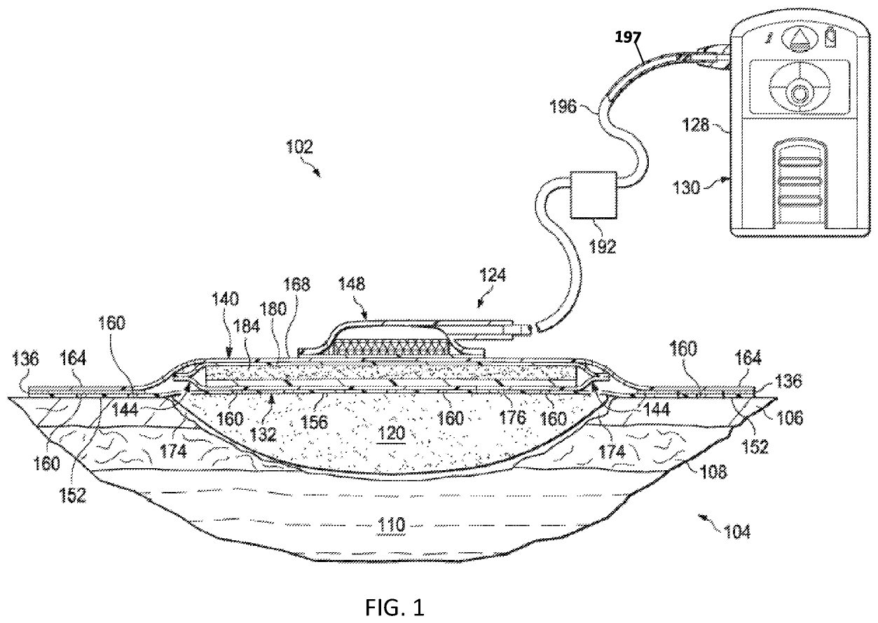

FIG. 1 illustrates a high-level side view of a therapy system 102 for treating a tissue site. Examples of such therapy systems are provided in U.S. patent application Ser. No. 15/356,063, filed on Nov. 18, 2016, the teachings of which are incorporated herein by reference in their entirety. Generally, the therapy system 102 can provide therapy to any portion of the tissue, such as the epidermis 106, the dermis 108, the subcutaneous tissue 110, or other tissue sites.

As shown in FIG. 1, the tissue site 104 can extend through or otherwise involve an epidermis 106, a dermis 108, and a subcutaneous tissue 110. For example, as shown in FIG. 1, the tissue site 104 can be a sub-surface tissue site that extends below the surface of the epidermis 106. Alternatively or additionally, the tissue site 104 can be a surface tissue site (not shown) that predominantly resides on the surface of the epidermis 106. For example, the tissue site 104 can be an incision site.

The therapy system 102 can include a dressing 124 and a reduced-pressure or a negative pressure source 128. The reduced pressure source 128 can be a component of an optional therapy unit 130. In some embodiments, the reduced-pressure source 128 and the therapy unit 130 can be separate components. A conduit 196 having an internal lumen 197 can be coupled in fluid communication between the reduced-pressure source 128 and the dressing 124.

An interface manifold 120 can be positioned proximate to or adjacent to the tissue site 104. The interface manifold 120 can be in fluid communication with the tissue site 104 to distribute reduced pressure to the tissue site 104. For example, the interface manifold 120 may be positioned in direct contact with the tissue site 104. The tissue interface or the interface manifold 120 can be formed from any suitable material available in the art. For example, the interface manifold 120 can be formed from any manifold material or flexible bolster material that provides a vacuum space, or treatment space (e.g., a porous and permeable foam or foam-like material, a member formed with pathways, a graft, or a gauze). In some embodiments, the interface manifold 120 can be a reticulated, open-cell polyurethane or polyether foam that is capable of being fluid permeable while under a reduced pressure.

The dressing 124 can include one or more layers, such as a base layer 132, an adhesive layer 136, and a sealing member 140. The dressing 124 can also include a fluid management assembly 144, and a conduit interface 148. Generally, the dressing 124 can be formed using any suitable number of layers and from any suitable material available in the art. Further, the dressing 124 can be configured to receive and/or provide a negative/reduced pressure from the reduced pressure source 128 to the interface manifold 120 to extract fluid from the tissue site 104 through the interface manifold 120.

The base layer 132 of the dressing 124 can be a soft, pliable material suitable for providing a fluid seal with the tissue site 104. Generally, the base layer 132 can be formed from any suitable material available in the art. For example, the base layer 132 can be a silicone gel, a soft silicone, hydrocolloid, hydrogel, polyurethane gel, polyolefingel, hydrogenated Styreniccopolymer gel, a foamed gel, a soft closed cell foam such as polyurethanes and polyolefins coated with an adhesive, polyurethane, polyolefin, or hydrogenated styrenic copolymers. The base layer 132 can also have any suitable thickness or stiffness. For example, the base layer 132 can have a thickness ranging between about 500 microns (Lm) and about 1000 microns (Lm). Further, the base layer 132 can have a stiffness ranging between about 5 Shore OO and about 80 Shore OO (e.g., in a range of about 19 shore OO to about 70 shore OO or in a range of about 20 shore OO to about 60 shore OO).

The base layer 132 can have a periphery 152 surrounding a central portion 156 and a plurality of apertures 160 disposed through the periphery 152 and the central portion 156. The apertures 160 in the base layer 132 can be in fluid communication with the interface manifold 120 and the tissue surrounding the tissue site 104. Further, the base layer 132. can have any number of corners 158, edges 159, and a border 161 that substantially surrounds the central portion 156 and is positioned between the central portion 156 and the periphery 152.

The adhesive layer 136 can be exposed to the apertures 160 in at least the periphery 152 of the base layer 132. For example, the adhesive layer 136 can be positioned adjacent to and/or positioned in fluid communication with the apertures 160 in at least the periphery 152 of the base layer 132. Further, the adhesive layer 136 can be exposed to or in fluid communication with tissue surrounding the tissue site 104 through the apertures 160 in the base layer 132. Generally, any suitable adhesive layer 136 can be used. For example, the adhesive 136 can comprise an acrylic adhesive, rubber adhesive, high-tack silicone adhesive, polyurethane, or other adhesive substance.

The sealing member 140 can have a periphery 164 and a central portion 168. The sealing member 140 can also include an aperture 170. The periphery 164 of the sealing member 140 can be positioned proximate to the periphery 152 of the base layer 132 such that the central portion 168 of the sealing member 140 and the central portion 156 of the base layer 132 define an enclosure 172. The adhesive 136 can be positioned at least between the periphery 164 of the sealing member 140 and the periphery 152 of the base layer 132. The sealing member 140 can cover the tissue site 104 and the interface manifold 120 to provide a fluid seal and a sealed space 174 between the tissue site 104 and the sealing member 140 of the dressing 124. Further, the sealing member 140 can cover other tissue, such as a portion of the epidermis 106, surrounding the tissue site 104 to provide a fluid seal between the sealing member 140 and the tissue site 104. The sealing member 140 can be formed from any suitable material that allows for a fluid seal. A fluid seal can be a seal adequate to maintain reduced pressure at a desired site given the particular reduced pressure source or system involved.

The fluid management assembly 144 can be disposed in the enclosure 172 defined by the central portion 168 of the sealing member 140 and the central portion 156 of the base layer 132. The fluid management assembly 144 can include a first dressing wicking layer 176, a second dressing wicking layer 180, and an absorbent layer 184 (or a dressing absorbent 184). The absorbent layer 184 can be positioned in fluid communication between the first dressing wicking layer 176 and the second dressing wicking layer 180. The first dressing wicking layer 176 can have a grain structure adapted to wick fluid along a surface of the first dressing wicking layer 176. Similarly, the second dressing wicking layer 180 can have a grain structure adapted to wick fluid along a surface of the second dressing wicking layer 180. For example, the first dressing wicking layer 176 and the second dressing wicking layer 180 can wick or otherwise transport fluid in a lateral direction along the surfaces of the first dressing wicking layer 176 and the second dressing wicking layer 180, respectively. The surface of the first dressing wicking layer 176 can be normal relative to the thickness of the first dressing wicking layer 176, and the surface of the second dressing wicking layer 180 can be normal relative to the thickness of the second dressing wicking layer 180. The wicking of fluid along the first dressing wicking layer 176 and the second dressing wicking layer 180 can enhance the distribution of the fluid over a surface area of the absorbent layer 184, which can increase absorbent efficiency and resist fluid blockages. Fluid blockages can be caused by, for example, fluid pooling in a particular location in the absorbent layer 184 rather than being distributed more uniformly across the absorbent layer 184. The laminate combination of the first dressing wicking layer 176, the second dressing wicking layer 180, and the absorbent layer 184 can be configured to maintain an open structure, resistant to blockage and capable of maintaining fluid communication with, for example, the tissue site 104.

The conduit interface 148 is configured such that it is in fluid communication with the dressing 124, through the aperture 170 in the sealing member 140, and can provide reduced pressure from the reduced-pressure source 128 to the dressing 124. The conduit interface 148 can also be configured to be positioned in fluid communication with the optional interface manifold 120. An optional liquid trap 192 may be positioned in fluid communication between the dressing 124 and the reduced-pressure source 128. The liquid trap 192 can be any suitable containment device having a sealed internal volume capable of retaining liquid, such as condensate or other liquids.

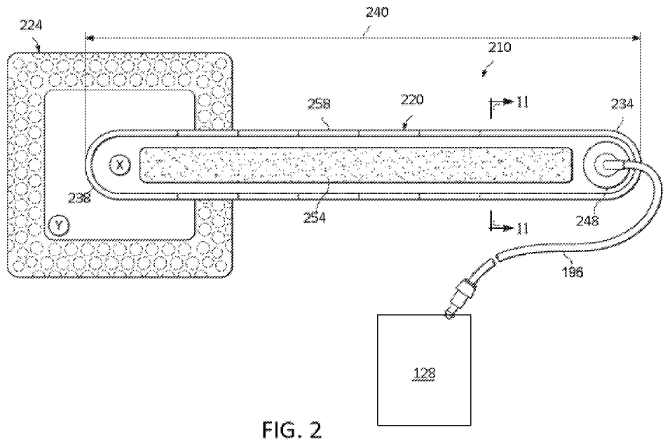

In some embodiments, a bridge assembly 210 can extend away from the tissue site 104 and the dressing 124 to define a fluid passageway between the tissue site 104 and the reduced-pressure source 128. The bridge assembly 210 can be configured such that it provides a fluid communication between the dressing 124 and the reduced-pressure source 128. As shown in FIG. 2, the bridge assembly 210 can include a bridge 220 and a conduit interface 248 that is configured to provide reduced pressure from the reduced-pressure source 128, through the bridge 200 to the dressing 224.

The bridge 220 can extend along a length 240 that separates and/or spaces apart a receiving end 234 of the bridge from its transmitting end 238. The receiving end 234 can have an aperture (not shown) that is in fluid communication with an aperture (not shown) on the transmitting end 238 of the bridge. The bridge 220 can further include one or more wicking layers 254 and a bridge sealing member 258. The bridge sealing member 258 can extend along the length 240 of the bridge 220.

The receiving end 234 of the bridge (e.g., through the aperture of the receiving end) can be fluidly connected to a conduit interface 248. The conduit interface 248 of the bridge can be configured to connect to a conduit 196 that connects the bridge 220 to a reduced-pressure source 128. Further, the conduit interface 248 of the bridge can be in fluid communication with the transmitting end 238 of the bridge through the length 240 of the bridge 220. The transmitting end 238 of the bridge can, in turn, be coupled to and/or in fluid communication with the dressing 224. Therefore, the conduit interface 248 of the bridge can fluidly connect the negative pressure source 128, through the conduit 196 and the length of the bridge 240, to the dressing 224.

As noted, the dressing 224 can be any suitable dressing available in the art. For example, the dressing 224 can be a specialized dressing that is configured for use in treating complex wounds, such as venous leg ulcers (VLU), etc. Additionally or alternatively, the dressing 224 can be specialized dressing configured for use in combination therapies. For example, the dressing 224 can be a dressing configured for use in NPWT and/or along with compression garments (e.g., bandages, garments, and stockings). Additionally or alternatively, the dressing 224 can be a dressing configured for use in extended care. For example, the dressing 224 can be a shallow wound dressing configured for providing therapy over an extended period of time (e.g., multiple days). In some embodiments, the dressing 224 can be a shallow wound dressing configured for providing extended therapy (e.g., seven day) using a mechanically driven device, such as a negative pressure device (NP device). For example, the dressing 224 can be used in conjunction with a wound therapy system 102, such as that described FIGS. 1-2. As described with reference to FIGS. 1-2, the wound therapy system 102 can include various elements including but not limited to a base wound dressing 224, a fluid management bridge assembly 210, and a conduit 196 (e.g., tube) for connection to a preferred NPWT device 128.

The fluid management bridge assembly 210 and the base wound interface dressing 224 are often supplied as separate components that are assembled by an end user (e.g., medical practitioner, such as a nurse or physician) at the wound site. This provides the end user with the freedom to orient the dressing 224 and/or fluid management bridge assembly 210 as needed. This is a valuable feature of such wound therapy systems, because it allows the end user to customize the dressing 224 and the fluid management bridge assembly 210 for the individual patient and/or application (e.g., customize to the location and size of the wound). However, customization of the dressing and fluid bridge placement can present a challenge to the end user because the end user may not always align and assemble these elements correctly. Improper alignment and assembly can, in turn, result in impeding the functional performance of the dressing. For example, improper alignment of these components can result in creation of a bottleneck that reduces the cross-sectional area of the fluid communication established between the dressing 224 and the fluid management bridge assembly 210. Further, improper alignment of the dressing 224 and the fluid management bridge assembly 210 can compromise the sealing feature between these components and result in possible leakage of fluids. Embodiments disclosed herein overcome the challenges and difficulties in establishing proper alignment among various components of wound therapy systems.

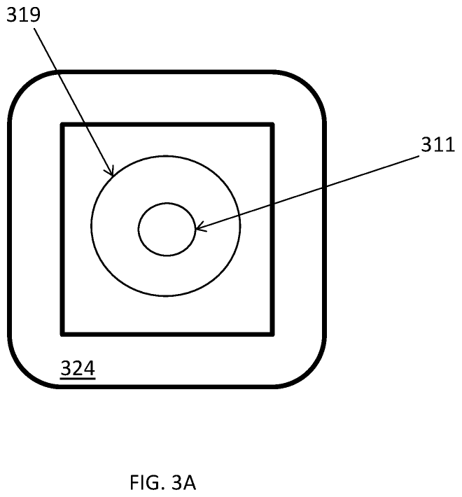

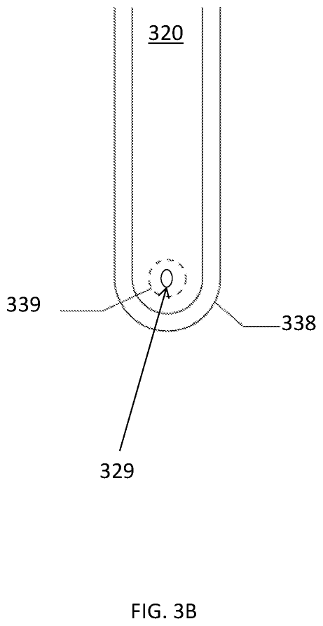

FIG. 3A illustrates a base wound dressing 324 according to some embodiments disclosed herein. As shown in FIG. 3A, the dressing 324 includes one or more alignment features 319 that are configured to facilitate connection of the fluid connection port 311 of the dressing 324 to fluid ports (e.g., a fluid port of a fluid management bridge assembly 210 (FIG. 2) or a fluid port of the conduit interface 148) of other portions of the wound therapy system 102. The alignment features 319 can be any suitable available features. For example, the alignment features 319 can be one or more geometric patterns.

The alignment features 319 can be configured such that they are adjacent to (e.g., centrally aligned) and/or are geometrically linked (e.g., dimensionally aligned) to the fluid connection port 311 of the dressing 324. Specifically, the alignment features 319 can be configured to surround, label, outline, highlight, mark, specify, and/or be adjacent to the fluid connection port 311 of the dressing. The alignment features 319, by specifying the location of the port 311, can indicate the approximate location of the fluid port 311 to the user (e.g., practitioner assembling/connecting the wound therapy system), thereby facilitating the connection of the fluid port 311 with fluid ports of other portions of the wound therapy system 102. The alignment features 319 can further be configured such that the correspond to alignment features included on other portions of the wound therapy system 102. For example, as shown in FIG. 3B, the transmitting end 338 of the bridge 320 of the wound therapy system can be configured to include one or more alignment features 339.

The one or more alignment features 339 of the bridge 320 can be configured such that they are adjacent to and/or are dimensionally linked to the fluid port 329 of the bridge 320. Specifically, the one or more alignment features 339 can be configured to surround, label, outline, highlight, mark, specify, and/or be adjacent to the fluid connection port 329 of the bridge 320. The alignment features 339, by specifying the location of the port 329, can indicate the approximate location of the fluid port 329 to the user.

As noted, the alignment features 319 of the dressing 324 can be configured such that they mate and/or complement to the alignment features 339 of the bridge 320. For example, the alignment features 319 of the dressing 324 and the alignment features 339 of the bridge 320 can be complementary and/or matching features. The matching and/or complementary alignment features can allow the users to match the alignment features 319 of the dressing 324 to the alignment features 339 of the bridge 320, thereby facilitating establishing a connection between the port 311 of the dressing 324 with the port 329 of the bridge 320.

The alignment features 319, 339 can be any suitable alignment features. For example, the alignment features 319, 339 can comprise one or more geometrical shapes including but not limited to at least one of: one or more circular shapes, one or more polygonal shapes, or a. combination thereof. In the example embodiments shown in FIGS. 3A-3B, circular alignment features 319, 339 that are disposed adjacent to the ports 311, 329 (and are dimensionally linked to the ports) are shown.

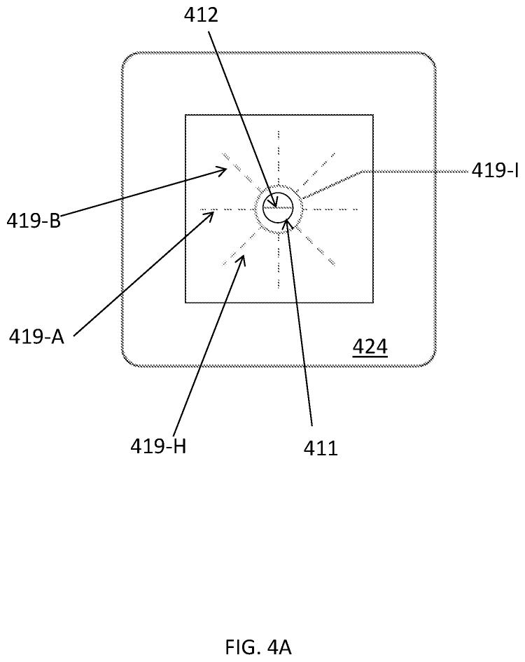

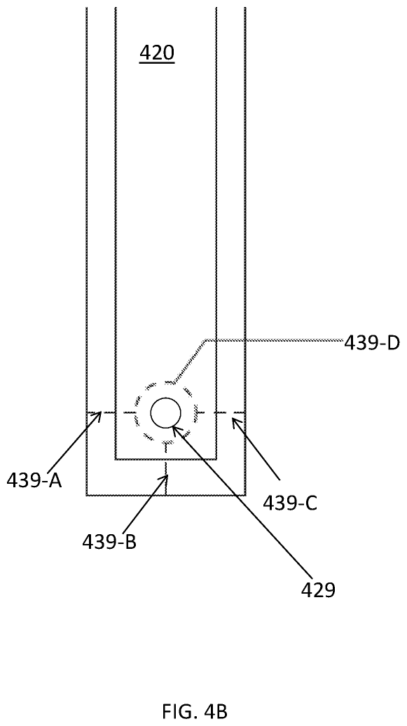

As noted, other suitable alignment features 319, 339 can be employed. For example, the alignment features 319, 339 can include one or more lines configured such that each line is perpendicular to a central axis line of the corresponding fluid port 311, 329. Specifically, as shown in FIG. 4A. the dressing 424 can include one or more alignment features 419-A, 419-B, . . . , 419-H configured as lines that extend along a central axis line 412 of the port 411 and/or are perpendicular to a central axis line 412 of the port 411 of the dressing 424.

As shown in FIG. 4B, the bridge 420 can also include complementary features as those shown on the dressing 424. Specifically, the bridge 420 can include one or more alignment features 439-A, . . . , 439-C that are configured to complement the alignment features 419-A, 419-B, . . . , 419-H of the dressing 424.

Although described as “complementary features,” it should be understood that the alignment features included on one element of the wound therapy system need not identically correspond to the alignment features included on other portions of the wound therapy system. Specifically, one element of the wound therapy system can include different and/or additional alignment features than other elements of the wound therapy system. For example, as shown in FIG. 4B, fewer alignment features 439-A, . . . , 439-C are included on the bridge 420 than those included 419-A, 419-B, . . . , 419-H on the dressing 424.

Further, various alignment features can be used in conjunction with one another. For example, as shown in FIGS. 4A-4B, geometric alignment features 419-1 (surrounding the fluid port 411 of the dressing 424) and 439-D (surrounding the fluid port 429 of the bridge 420) can be used in combination with other alignment features (e.g., lines 419-A, 419-B, , 419-H on the dressing 424 and lines 439-A, . . . , 439-C on the bridge 420) to facilitate establishing an alignment between the dressing 424 and the bridge 420.

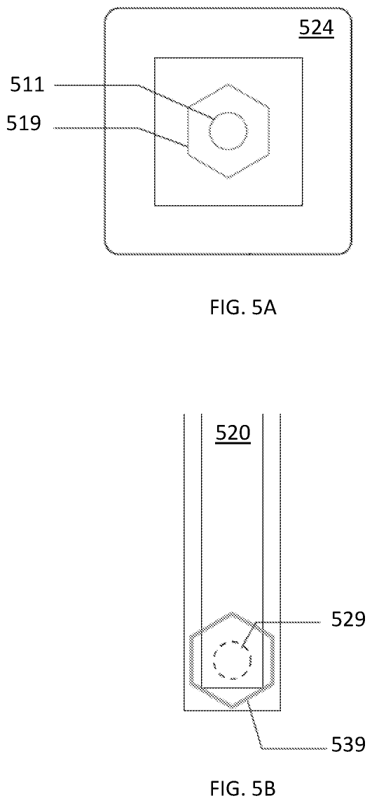

As noted, the alignment features can assume various shapes and forms. For example, as shown in FIG. 5A, the dressing 524 can include one or more geometrical (e.g., hexagonal-shaped) features 519 that are geometrically linked with and/or disposed adjacent to the fluid port 511 of the dressing 524. As shown in FIG. 5B, the bridge 520 can include complementary alignment features 539 that are configured to facilitate establishing an alignment between the dressing 524 and the bridge 520. Specifically, as shown in FIG. 5B, the bridge 520 can include one or more geometrical (e.g., hexagonal-shaped) features 539 that are geometrically linked with and/or disposed adjacent to the fluid port 529 of the bridge 520.

Although described as matching and/or complementary alignment features, the alignment features 539 of the bridge 520 and the alignment features 519 of the dressing 524 need not to be matching and/or complementary. Generally, any alignment feature that surrounds, labels, outlines, highlights, marks, specifies, and/or is adjacent to a fluid connection port (e.g., fluid connection port 529 of the bridge 520 or fluid connection port 511 of the dressing 524) can be employed. Additionally or alternatively, one or more components of the wound therapy system can include one or more alignment features while other elements of the wound therapy system do not include any alignment features. For example, in some embodiments, the dressing can include one or more alignment features while the bridge does not include any alignment features. Further, various components of the wound therapy system need not include the same number of alignment features. Specifically, one or more components of the wound therapy system can include a certain number of alignment features while other elements of the wound therapy system include more or fewer alignment features. For example, in some embodiments, the dressing can include a certain number (e.g., five) alignment features while the bridge includes a fewer number of alignment features (e.g., three).

As noted any suitable alignment feature can be used. For example, the alignment features can include one or more geometric patterns or other shapes, designs, dimensions, and/or patterns. These alignment features can be dimensionally linked to the location of the location of the fluid connection ports on various components of the wound therapy system, including the wound interface dressing 524 and the fluid management bridge 520. The alignment of these geometric shapes during the dressing application can be used to ensure the proper alignment of the fluid connection ports on both the base wound interface dressing 524 and the fluid management bridge 520. The positive feedback of aligning a geometric pattern can be used to provide assurance to the user that the dressing system has been applied correctly before commencing further with the therapy regime.

Further, as noted above, alignment features can comprise circular shapes or geometric patterns comprising circular shapes. The circular shapes can be centrally aligned or dimensionally linked to the fluid connection port location on the base wound interface dressing. Circular alignment features can allow for 360° rotation of the bridge while maintaining proper alignment of the fluid connection ports. As noted, the fluid management bridge can have complementary or different alignment features. The diameter of circular features included on the dressing can be selected based on the shape or pattern of the features included on the fluid management bridge. For example, depending on the geometric pattern used on the fluid management bridge, the size and/or diameter of the alignment features included on the dressing can be varied to ensure proper alignment of the fluid management bridge and the dressing (e.g, for example, if the fluid management bridge includes a hexagonal alignment feature, the dressing can include a circular feature having a diameter equal to or greater than the largest axes of the hexagon).

Furthermore, as noted previously, the alignment features can comprise one or more “cross hair” center line axes that run through the center of a fluid connection port (e.g., as shown in FIG. 4A, one or more cross hair lines that run through the center of the fluid connection port of the dressing.). Such alignment features can comprise two singular axes running through the central fluid connection port, equally spaced at 90° intervals from each other, or four axes running through the central fluid connection port, equally spaced at 45° intervals from each other, etc.

Still further, as noted, alignment features can comprise one or more polygonal shapes. In some embodiments, polygonal shapes having an even number of sides (e.g., square, hexagon, octagon, etc.) can be used. The polygonal shapes can be configured such that they are centrally aligned and/or dimensionally aligned with their corresponding fluid port (e.g., fluid port of the dressing if used on the dressing). In some embodiments, the dressing and/or the fluid bridge can include multiple alignment features in an effort to increase the number or alignment positions. For example, in the example shown in FIG. 4A-4B, the dressing includes both an circular alignment feature and multiple cross hair alignment features 419A, . . . , 419I, and each of these alignment features can be used, independent of other alignment features, to facilitate establishing a connection between the dressing and the fluid bridge.

As noted with reference to FIGS. 3A-5B, the alignment features can include (but are not limited to) any of circular shapes, polygonal shapes, and/or center line axis (cross hair) shapes. The circular shapes can be concentrically aligned or dimensionally linked to the fluid connection port location on the base wound interface dressing. The circular shapes can allow for 360° rotation of the fluid management bridge while maintaining proper alignment of fluid connection ports. The circular shapes can comprise any suitable diameter and the diameter selected for the circular shapes can vary depending on the shape and size of the mating alignment features included on the fluid management bridge.

Generally, any polygonal shape can be used with the embodiments disclosed herein. For example, polygonal shapes having an even number of sides can be employed. The polygonal shapes that can be used with the embodiments disclosed herein include but are not limited to squares, hexagons, octagons, etc. The polygonal features can be centrally and/or dimensionally aligned with the fluid communication port on the base wound interface dressing. In some embodiments, the base dressing can include two or more alignment features, having different shapes and/or sizes, in an effort to increase the number of alignment positions that can be achieved. For example, as shown in FIG. 5C, the dressing 524 can include one or more circular features 519A, 519B, one or polygonal features octagonal features 519C and hexagonal features 519D), and one or more square or diamond shapes features 519E, 591F, 519G that have been centrally and dimensionally aligned with the fluid port 511 of the base wound dressing 524. Further, the base wound dressing 524 can include one or more linear (cross hair) features 519-H, 519-I, 519-J, 519-K. As noted previously. the cross hair features can extend along center line (e.g., axis line) of the fluid port 511 of base wound dressing 524. For example, the cross hair alignment features 519-H, 519-I, 519-J, 519-K can comprise singular axis lines extending through the central fluid connection port 511. The cross hair alignment features can be positioned at any suitable angle with respect to one another. For example, in one embodiment, two cross hair lines equally spaced at 90° intervals from one another can be used. Alternatively or additionally, in some embodiments, four axis lines running through the central fluid port 511 and equally spaced at 45° intervals from one another can be used. One of ordinary skill in the art should appreciate that although described with reference to base wound dressing 524, similar alignment features and alignment feature configurations can be employed on the fluid management bridge.

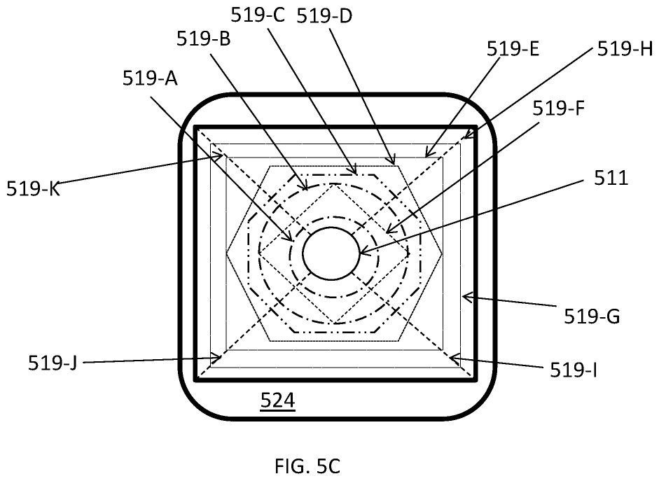

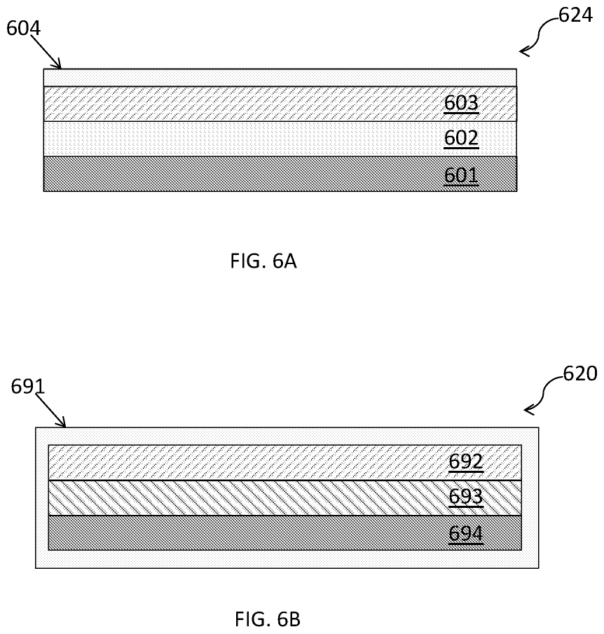

The base dressing can be generally formed from any suitable material. For example, as shown in FIG. 6A, the base dressing 624 can be formed using one or more layers of various materials. For example, the dressing 624 can be comprised of 1) a patient interface layer 601, 2) two or more wicking layers 602, 603, and 3) a base adhesive layer 604. In some embodiments, the patient interface layer 601 can comprise a material such as a silicone or perforated silicone. Further in some embodiments, the base adhesive layer 604 can be an occlusive adhesive coated top layer, for example an occlusive adhesive Polyurethane (FU) top layer. The one or more wicking layers 602, 603 can comprise any material capable of capturing, distributing, transferring, and/or storing fluid. Although not shown in FIG. 6A, the dressing 624 can also include one or more absorbent layers.

Similarly, as shown in FIG. 6B, the fluid management bridge 620 can be formed from any suitable material. For example, the fluid management bridge 620 can be formed using one or more layers of various materials. In some embodiments, the fluid management bridge 620 can include an outer shell or housing 691 that is configured to house the internal layers or components of the fluid management bridge 620. The outer shell 691 can comprise any suitable material. For example, the outer shell can comprise Polyurethane (PU) film. The fluid management bridge 620 can further comprise one or more middle layers 692, 693. The middle layers can include one or more wicking layers and/or one or more absorbent layers. The one or more wicking layers and/or absorbent layers can comprise any material capable of capturing, distributing, transferring, absorbing, and/or storing fluid. Additionally or alternatively, the fluid management bridge 620 can include one or more absorbent components 694 (e.g., any suitable super absorbent component available in the art). For example, in some embodiments, the fluid management bridge 620 can include one or more super absorbent components 694 configured to store fluids within the fluid management bridge 620 for a predetermined period of time.

The alignment features described herein can be included in the base dressing 624 and/or the fluid management bridge 620 by inclusion on any suitable portion (e.g., any suitable layer) of these components. For example, the alignment features can be included in the base dressing 624 and/or the fluid management bridge 620 using any suitable scheme. For example, the alignment features can be applied by any of rotary printing or pad printing to the base dressing 624 and/or the fluid management bridge 620, Alternatively or additionally, the alignment features can be patterned coated as a part of an adhesive coating (e.g., base adhesive layer 604) included in the base dressing 624 and/or the outer shell 691 fluid management bridge 620. Further, the alignment features can be included in an ink, for example a biocompatible ink. Additionally or alternatively, the alignment features can be embossed, laminated, and/or be formed as an integral portion of the adhesive coated top layer (e.g., occlusive adhesive coated top layer) of the base wound dressing and/or the outer shell 691 of the fluid management bridge 961. As noted, in some embodiments, the alignment features can be included by pad/rotary printing and/or be pattern coated as a part of the coating (e.g., adhesive coating). Alternatively or additionally, the alignment features can be formed as a part of other parts of the base wound dressing 624 or the fluid management bridge 620 (e.g., by inclusion in the wicking or absorbent layers 692, 693, 694). Further, the alignment features can be embossed, edge welded, applied using heat (heat stakes), or a combination thereof.

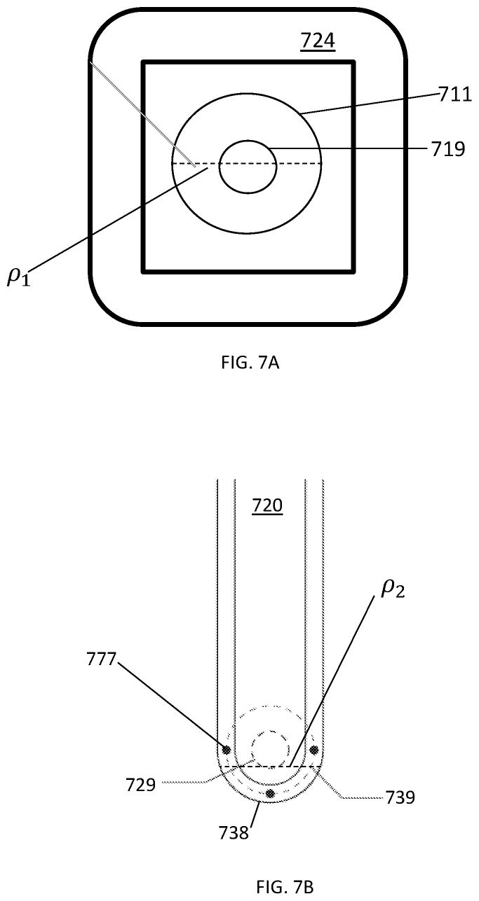

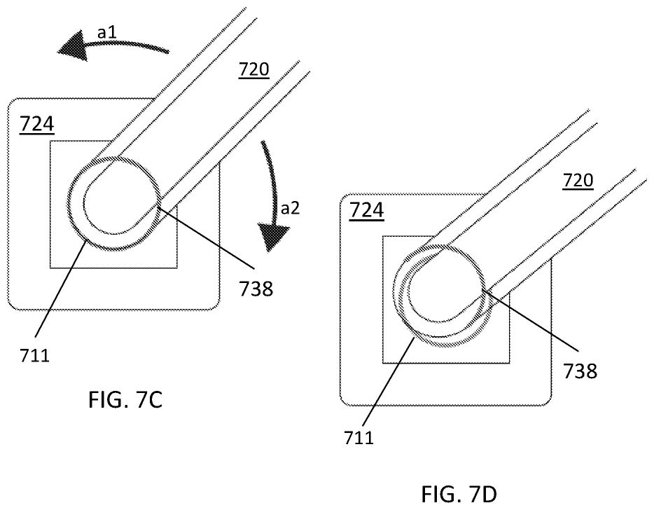

As shown in FIGS. 7A-7B, the transmitting end 739 of the fluid management bridge 720 can also be configured to serve as an alignment feature. Specifically, the transmitting end 729 of the fluid management bridge 720 can be configured to mate with the fluid port 719 of the base wound dressing 724 and/or alignment features included on the base wound dressing 724. For example, as shown in FIGS. 7A-7B, the transmitting end 738 of the fluid management bridge (e.g., the outer shell of the fluid management bridge 738) can be configured to comprise a semicircular shape and/or semicircular edge. The center of this semicircular edge can be configured to centrally align with the fluid communication port 729 of the bridge 720. This semicircular shape can further be configured to mate with corresponding alignment features 711 formed on the base wound dressing 724.

In some embodiments, to facilitate mating of the alignment bridge 720 with the base wound dressing 724, the diameter, ρ₁, of the alignment feature 711 of the base wound dressing 724 and the diameter, ρ₂, of the transmitting end 739 can be configured to be substantially equal. This can allow the user to align the edge of the fluid management bridge 738 with the alignment features 711 on the dressing 724 (FIG. 7C). Further, as shown in FIG. 7C, the user has the option of moving the alignment bridge 720, along the directions shown with arrows a1, a2, while connecting the fluid management bridge 720. As noted, this provides the end user with the freedom to orient the dressing 724 and/or fluid management bridge assembly 710 as needed. This can a valuable feature of such wound therapy systems, because it allows the end user to customize the dressing 724 and the fluid management bridge 720 for the individual patient and/or application (e.g., customize to the location and size of the wound).

As shown in FIG. 7C, the alignment features 711, 738 can facilitate forming a connection between the dressing 724 and the fluid management bridge 720 because the user can easily align the curved edge of the fluid management bridge 720 to the alignment features of the dressing 724 to establish a connection between these components.

It should be noted that although shown as circular alignment features 711, 738, embodiments disclosed herein are not limited to the use of circular features. The transmitting end 738 of the fluid management bridge 720 can assume any shape or form (e.g., polygonal shape) that can facilitate making a connection between the fluid management bridge 720 and the dressing 724. The shape and form of the fluid management bridge 720 can be implemented in the outer shell of the bridge at the transmitting end 738 using any suitable technique. For example, the semicircular shape shown in FIGS. 7A-7D can be formed as a part of the fluid management bridge 720 contents or shaped within the edge weld.

In some embodiments, the edge of the transmitting end 738 can be continued in print to complete the shape. For example, the semicircular shape of the fluid management bridge 720 can be continued in print to complete the circle. For example, as shown in FIG. 7C, the circle can be completed by the edge of the bridge and the print. The completed printed shape can further assist in connecting the fluid management bridge 720 to the dressing 724.

FIG. 7D schematically illustrates an example of a case in which the alignment features 711 of the dressing 724 and the transmitting end 738/alignment features of the fluid management bridge 720 are not fully aligned. As shown, the alignment features allow the user to observe that the fluid port of the dressing and the transmitting end 738 of the fluid management bridge are not properly/adequately connected and facilitate forming a proper connection between these components.

The shape of the transmitting end 738 of the fluid management bridge 720 does not limit the fluid management bridge 720 for use with dressings 724 having similar alignment features. Generally, any fluid management bridge 720, having any alignment feature, can be used with any dressing 724, having any alignment feature. For example, the semicircular shaped fluid management bridge 720 can be used with dressings 724 having polygonal or cross hair alignment features (e.g., dressing 724 shown in FIG. 5C). For example, referring to dressing 524, shown in FIG. 5C, the semicircular transmitting end 738 can be used in conjunction with any of the alignment, features shown in FIG. 5C. Specifically, the user can select any of the alignment features (e.g., diamond 519-F) that can match the transmitting end 738 or fully cover the circle formed by transmitting end 738 of the bridge. For example, the user can check the alignment features 519-A, . . . , 519-J to determine if any of the alignment features have two or more sides that would be tangent to the perimeter of the circle formed by the transmitting end 738. Alternatively or additionally, the user can use the cross hair features 519-H, . . . , 519-J and place the transmitting end 738 such that the cross hair features 519-H, . . . , 519-J are disposed along the diagonal axes of the circle formed by the transmitting end 738.

Further, as shown in FIG. 7B, the fluid management bridge 777 can include one or more indication points 777 that are configured to be concentric with the fluid port 729 of the fluid management bridge 720. In some embodiments, the indication points 777 can be configured such that they are disposed along a circle having a similar circular print as circular alignment features (or cross hair features) included on the dressing 724. The alignment of the indication points 777 with the circular alignment features (or cross hair features) on the dressing 724 can further ensure the user that a proper connection between the fluid management bridge 720 and the dressing 724 is established. It should be noted that the indication points 777 can define any shape, for example a polygonal shape. The polygonal alignment features can be configured such that alignment of three or more sides of the polygonal features indicates that a. proper connection between the fluid management bridge 720 and the dressing 724 is established.

As noted, the transmitting end 738 of the fluid management bridge can assume any shape and form. For example, the transmitting end 738 of the fluid management bridge can have a square end. Such alignment feature can be used to mate the fluid management bridge 720 with polygonal alignment features (e.g., having even number of sides) on the base wound dressing 724. Further, the end of the fluid management content, the outer shell of the fluid management bridge, the edge welds of the fluid management bridge, and/or alignment features included on the transmitting end of the fluid management bridge can serve as alignment features for the fluid management bridge. For example, in embodiments that utilize weld lines as alignment features, the user can align the weld lines to ensure that at least three edges of the weld lines are aligned with alignment features on the dressing 724. This can ensure proper alignment of the fluid connection ports.

Similarly, in embodiments that utilize polygonal alignment features, alignment features can be configured such that proper alignment can be achieved when three or more sides of a polygon on the base wound dressing is aligned with three or more sides of a similarly sized polygon on the fluid management bridge. Further, the cross hair alignment features can be configured similarly to ensure that once two or more cross hair features (e.g., two cross hair features positioned 90° apart or three cross hair features positioned 45° apart) from the fluid management bridge are aligned with two or more corresponding cross hair features, a proper connection between the fluid ports of the fluid management bridge and the base wound dressing is established.

Alternatively or in addition to being included on base wound dressings and/or the fluid management bridge, the alignment features disclosed herein can be included in conventional dressing pads, such as interface pads or an independently applied dressing, to allow the customization of the arrangement of base components to the individual patient needs. In such applications, proper alignment can be important for ensuring peak system performance. For example, the alignment features can be included on an occlusive adhesive drape included on a wound filler supplied to a user (e.g., a wound filler that the user can shape as required). In some embodiments, the drape can have a pre-cute aperture or hole and include one or more of the alignment features described herein. The interface pad or bridge can have corresponding markings to allow proper alignment of fluid connection ports.

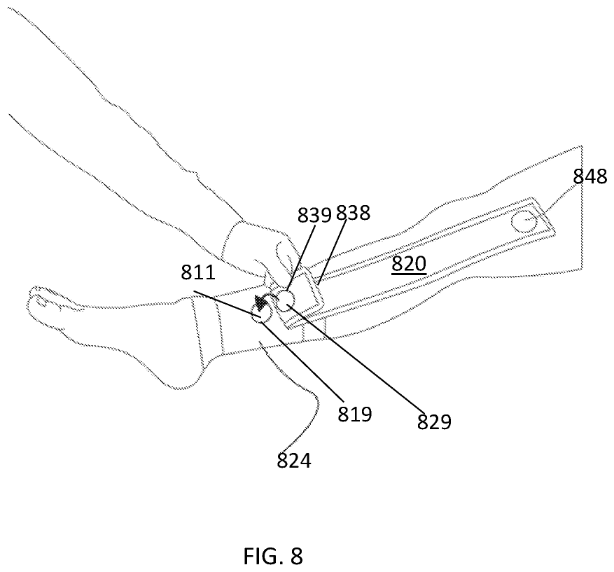

FIG. 8 graphically illustrates a wound therapy system according to some embodiments disclosed herein. As shown, fluid can be extracted from a base wound interface 824 by aligning one or more alignment features 839 surrounding the fluid port 829 disposed on a transmitting end 838 of the fluid management bridge 820 with corresponding alignment features 819 surrounding a fluid port 811 of the base wound interface 824. Once a connection between the two fluid ports 811, 829 is established, fluid can be extracted from the base wound interface 824. As described with reference to FIGS. 1-2, the fluid management bridge 820 can comprise a conduit interface 848 of the bridge that is configured to connect the bridge 820 to a reduced-pressure source.

The alignment features disclosed herein provide an end user with the ability to place a dressing or a wound cover over a wound without having to manage the orientation of the bridge component. This allows the user to achieve a proper seal between the components of the wound management system.

While the invention has been particularly shown and described with reference to specific illustrative embodiments, it should be understood that various changes in form and detail may be made without departing from the spirit and scope of the invention. Further, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

1. A wound dressing system comprising:
a base wound interface configured to be disposed on a portion of a patient's skin;
and
a fluid management bridge, the fluid management bridge having a first end configured to be coupled to the base wound interface and a second end configured to connect to a negative pressure source;
wherein the first end of the fluid management bridge includes one or more alignment features configured to be aligned to corresponding alignment features on the base wound interface.

2. (canceled)

3. The wound dressing system of claim 1, wherein the first end of the fluid management bridge includes a first port configured to be coupled to a fluid port on the base wound interface and the second end of the fluid management bridge includes a second port configured to be coupled to the negative pressure device.

4. (canceled)

5. (canceled)

6. The wound dressing system of claim 3, wherein the one or more alignment features on the fluid management bridge comprise one or more lines, wherein each line perpendicular to a central axis line of the first port.

7. (canceled)

8. The wound dressing system of claim 6, wherein the corresponding alignment features on the base wound interface are disposed adjacent to the fluid port of the base wound interface.

9. (canceled)

10. The wound dressing system of claim 6, wherein the corresponding alignment features on the base wound interface comprise one or more lines, wherein each line is an extension of a central axis line of the fluid port.

11-13. (canceled)

14. The wound dressing system of claim 1, wherein the base wound interface comprises an adhesive layer configured to affix the base wound interface to the patient's skin

15. The wound dressing system of claim 14, wherein the corresponding alignment features are included on the adhesive layer of the base wound interface.

16. The wound dressing system of claim 15, wherein the corresponding alignment features are included on the adhesive layer by at least one of: applying a biocompatible ink, printing onto the adhesive layer of the base wound interface, embossing onto the base wound interface, or laminating on the base wound interface.

17. The wound dressing system of claim 1, wherein the fluid management bridge comprises an outer layer, a middle layer, and an inner layer.

18. The wound dressing system of claim 17, wherein the one or more alignment features are included on the outer layer of the fluid management bridge.

19. The wound dressing system of claim 18, wherein the one or more alignment features are included on the first end, over the outer layer of the fluid management bridge by at least one of: applying a biocompatible ink, printing onto the outer layer, embossing onto the outer layer, or laminating on the outer layer.

20-23. (canceled)

24. The wound dressing system of claim 18, wherein the one or more alignment features are included on at least one of the middle layer or the inner layer of the fluid management bridge.

25. A fluid management bridge for providing a fluid communication between a wound interface dressing and a negative pressure source, the fluid management bridge comprising:
a first end having a first fluid port configured to be coupled to a port of the wound interface dressing; and
a second end having a second fluid port configured to be coupled to the negative pressure device;
wherein the first end of the fluid management bridge is configured to provide for central alignment of the first end with the port of the wound interface dressing.

26. The fluid management bridge of claim 25, wherein the first end of the fluid management bridge is geometrically shaped to provide for the central alignment of the first end with the port of the wound interface dressing.

27. The fluid management bridge of claim 25, wherein the first end of the fluid management bridge comprises a semi-circular geometrical structure configured to centrally align with one or more alignment features on the base wound interface.

28. The fluid management bridge of claim 27, wherein the one or more alignment features are dimensionally linked to the port of the wound interface dressing.

29. The fluid management bridge of claim 27, wherein the one or more alignment features comprise one or more geometrical shapes including at least one of: one or more circular shapes, one or more polygonal shapes, or a combination thereof.

30. A method for extracting fluid from a base wound interface, the method comprising:
aligning one or more alignment features surrounding a first port disposed on a first end of a fluid management bridge with corresponding alignment features surrounding a fluid port of the base wound interface;
establishing a connection between the first port and the fluid port; and
extracting the fluid from the base wound interface, through the fluid port of the base wound interface and the first port of the fluid management bridge.

31. The method of claim 30, further comprising connecting a second port disposed on a second end of the fluid management bridge to a negative pressure device.

32. The method of claim 31, further comprising transferring the fluid extracted from the base wound interface to the negative pressure device through the fluid management bridge.

33. The method of claim 31, further comprising storing the fluid extracted from the base wound interface in the fluid management bridge.

34. (canceled)
